(19) United States
(12) Patent Application Publication
(10) Pub. No.: US 2026/0345033 A1
(43) Pub. Date: Nov. 5, 2026

(54) DETECTING CONDITIONS USING A CHEST-MOUNTED APPARATUS

(71) Applicant: Stryker Corporation, Portage, MI (US)

(72) Inventors: Robert Greenewald, Snohomish, WA (US); Robert G. Walker, Seattle, WA (US); Clinton T. Siedenburg, Everett, WA (US)

(73) Assignee: Stryker Corporation, Portage, MI (US)

(21) Appl. No.: 19/663,113

(22) Filed: Apr. 29, 2026

Related U.S. Application Data
(60) Provisional application No. 63/797,842, filed on Apr. 30, 2025.

(51) Int. Cl. A61B 5/113; A61B 5/00; A61B 5/083; A61B 5/1455

(52) U.S. Cl. CPC A61B 5/113; A61B 5/0002; A61B 5/0836; A61B 5/14551; A61B 5/6823; A61B 5/7275; A61B 2562/0219; A61B 2562/04

(57) ABSTRACT

An example method includes detecting, by a chest-mounted device, an acceleration at a first side of a chest of a subject; and detecting, by the chest-mounted device, an acceleration at a second side of the chest of the subject. The first side of the subject is divided from the second side of the subject by the subject's midline. The example method further includes determining a discrepancy between the acceleration at the first side of the chest and the acceleration at the second side of the chest; and determining a physiological condition or a treatment condition of the subject by analyzing the discrepancy.

### CROSS-REFERENCE TO RELATED APPLICATION

This application claims priority to U.S. Provisional App. No. 63/797,842, which was filed on Apr. 30, 2025 and is incorporated by reference herein in its entirety.

### BACKGROUND

Medical devices monitor conditions of subjects, such as patients, using sensors. Examples of sensors include electrodes configured to detect electrocardiograms (ECGs) and transthoracic impedances, blood pressure cuffs configured to detect blood pressures, oximeters configured to detect pulse oxygenations, and capnography sensors configured to detect capnographs. In many cases, medical devices utilize sensors that are configured to detect systemic physiological parameters of subjects. Moreover, the sensors are typically connected to medical devices via wired connections.

### BRIEF DESCRIPTION OF THE DRAWINGS

FIG. 1 illustrates an example environment for detecting device and physiological conditions.

FIG. 2 illustrates example signaling for determining a condition.

FIG. 3 illustrates example signaling for reporting one or more parameters.

FIG. 4 illustrates an example process for determining a condition of a subject.

FIG. 5 illustrates an example process for communicating parameters.

FIG. 6 illustrates an example of an external defibrillator configured to perform various functions described herein.

FIG. 7 illustrates a chest-mounted apparatus configured to perform various functions described herein.

### DETAILED DESCRIPTION

Various implementations described herein relate to detecting parameters and conditions of a subject using a chest-mounted apparatus. In some aspects, the chest-mounted apparatus is equipped with sensors configured to detect parameters of the subject and/or of the chest-mounted apparatus itself. In some implementations, the chest-mounted apparatus reports the parameters to an external device (e.g., an external medical device) configured to analyze a condition of the subject. In some implementations, the chest-mounted apparatus infers a condition of the subject, a condition of a treatment administered to the subject, or a condition of the chest-mounted apparatus based on the parameters.

Previous sensor devices were configured to detect systemic parameters of a subject, and were not capable of detecting differences between different parts of the subject's body. As a result, these sensors could not detect physiological conditions that impacted different parts of the subject's body in different ways. In particular implementations of the present disclosure, the chest-mounted apparatus includes sensors disposed on different sides of the chest of the subject. For instance, the chest-mounted apparatus includes motion sensors (e.g., accelerometers) that are symmetrically disposed on opposite sides of the subject's midline. In various implementations, these sensors enable the chest-mounted apparatus to detect conditions associated with uneven chest movement, such as pneumothorax, improperly placed airway adapters (e.g., endotracheal tubes), and chest compressions applied away from the subject's midline.

Previous sensor devices often relied on wired connections to convey data to a monitor or other medical device. However, when the monitor was connected to multiple sensor devices, configured to detect respective parameters, using individual wires, those wires could become tangled and could otherwise impede care. Various implementations of the present disclosure provide a combination device that detects multiple parameters of a subject and reports those parameters wirelessly in substantially real time. In various examples, the chest-mounted apparatus is configured to wirelessly communicate with the external device. In some cases, the chest-mounted apparatus is configured to transmit communication signals encoding various data packets indicative of parameters detected by the chest-mounted apparatus. In particular cases, individual data packets include data indicative of multiple parameters detected at substantially the same time. For instance, the data representing the parameters are interleaved together within the data packets. By combining data from different parameters into a single data packet, the chest-mounted apparatus can report time-aligned parameters efficiently and substantially in real-time to the external device. In various cases, the data is encrypted within the communication signals to protect sensitive data from exposure during wireless transmission.

Various implementations of the present disclosure will now be described with reference to the accompanying figures.

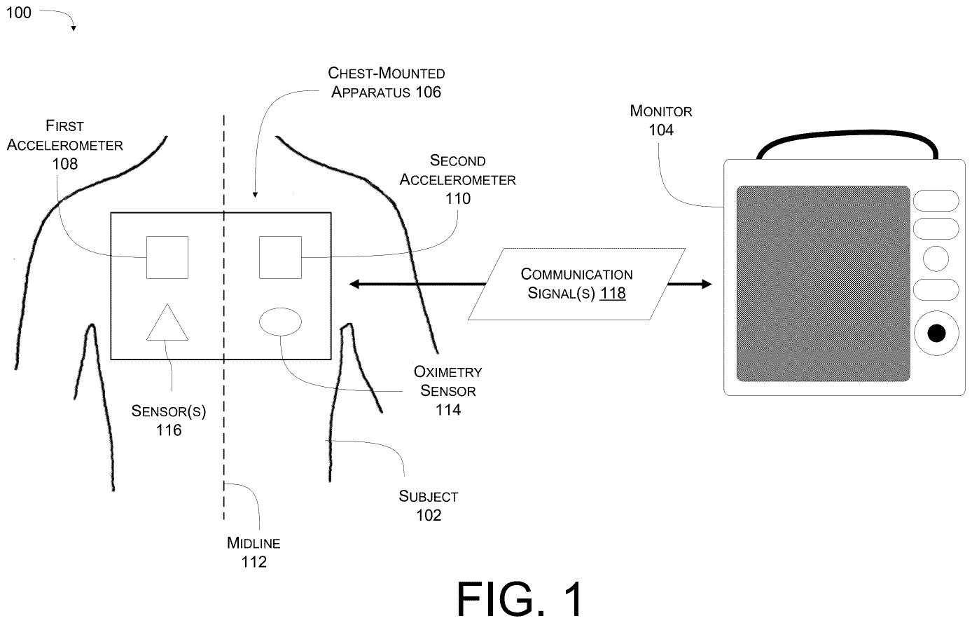

FIG. 1 illustrates an example environment 100 for detecting device and physiological conditions. The environment 100 includes a subject 102 experiencing an acute medical emergency. In some examples, the subject 102 has suddenly lost consciousness within the environment 100. The environment 100, in some instances, is a non-clinical environment. For instance, one or more rescuers are deployed to the environment 100 to attend to the condition of the subject 102 prior to transporting the subject 102 to a clinical environment, such as a hospital.

In various cases, a monitor 104 is utilized to monitor and/or treat the subject 102. The monitor 104 may be a portable medical device configured to function in the environment 100. In some examples, the monitor 104 can be physically carried to the environment 100 by one or more rescuers. The monitor 104, for instance, includes a rechargeable battery that is configured to power the monitor 104.

The monitor 104 includes, or is communicatively coupled with, one or more accessories including sensors configured to detect one or more physiological parameters of the subject 102. Examples of the sensors include electrodes (e.g., electrodes configured to be disposed externally on skin of the subject 102, pulse sensors, blood flow sensors (e.g., an ultrasound transducer configured to perform Doppler-based measurements of blood velocity), imaging devices (e.g., an ultrasound transducer configured to perform imaging of internal structures within the body of the subject 102, an oximetry device, a gas sensor, a pressure sensor, an airflow sensor, a thermometer, or any combination thereof. Examples of the physiological parameters include an electrocardiogram (ECG), an electroencephalogram (EEG), an electromyogram (EMG), an electrical impedance (e.g., a transthoracic impedance), a heart rate, a pulse rate, a blood flow parameter (e.g., a velocity or volumetric flow rate of blood through one or more blood vessels), a blood vessel shape (e.g., a cross-sectional area of one or more blood vessels), a blood oxygenation (e.g., a pulse oxygenation (SpO₂), a regional oxygenation, a cerebral oxygenation, a photoplethysmography, etc.), an airway parameter (e.g., a flow of air in an airway, a pressure of air in the airway, a partial pressure of one or more gasses (e.g., CO₂, O₂, etc.) in the airway, a respiration rate, a ventilation rate, etc.), a capnograph, an end-tidal CO₂ (EtCO₂), a blood pressure (e.g., a systolic blood pressure, a diastolic blood pressure, an instantaneous blood pressure, etc.), a temperature (e.g., a core temperature), or any combination thereof. In various cases, various individual accessories may be connected to the monitor 104 over one or more wired connections.

In various cases, the monitor 104 evaluates a condition of the subject 102 by analyzing the physiological parameter(s). For instance, the monitor 104 is configured to determine whether the ECG of the subject 102 is indicative of a shockable arrhythmia, such as ventricular fibrillation (VF). The monitor 104 may include an output device, such a display, configured to present the detected physiological parameter(s) and/or condition.

In some examples, the monitor 104 further includes treatment capabilities. For instance, the monitor 104 may be configured to administer an electrotherapy (e.g., an electrical shock, pacing pulses, etc.) to the subject 102 to treat the condition of the subject 102. In some examples, the monitor 104 outputs (e.g., on the display) a recommendation to apply a treatment to the subject 102 in response to detecting the condition.

In various implementations, the monitor 104 is a portable medical device. However, the monitor 104 may be unsuitable for wearing by the subject 102, such as due to the size and weight of its input devices, internal circuitry (e.g., capacitor configured to output an electrotherapy), and other components. Accordingly, the accessories may be separate from the monitor 104 and communicatively coupled with the monitor 104 over various removably coupled, wired connections. Some accessories, such as blood pressure cuffs and gas (e.g., capnography) sensors, are also connected to the monitor 104 via hoses and other types of air channels. In high-stress, urgent, and complex rescue scenes, such connections can cause problems related to caring and transporting the subject 102. For instance, the wired connections can get tangled, making it difficult for a rescuer to maintain couplings of the connections to the subject 102 and/or monitor 104. In addition, the wired connections can impede access to the subject 102. For instance, various wired connections can impede the rescuer from administering chest compressions, assisted ventilation, or airway management of the subject 102.

In various implementations of the present disclosure, a chest-mounted apparatus 106 with sensing capabilities is wirelessly coupled with the monitor 104. The chest-mounted apparatus 106 is configured to be adhered, attached, or otherwise disposed on the chest of the subject 102. For instance, the chest-mounted apparatus 106 may include a housing that is adhered to skin on the chest of the subject 102. In some cases, the chest-mounted apparatus 106 includes one or more straps, buckles, elastic bands, or other fasteners configured to hold the chest-mounted apparatus 106 at a fixed position relative to the skin on the chest of the subject 102. In various cases, the housing of the chest-mounted apparatus 106 is wearable.

In some cases, the housing of the chest-mounted apparatus 106 enables the monitor 104, other devices, and rescuers to administer therapies to the subject 102 while the chest-mounted apparatus 106 is disposed on the chest of the subject 102. For example, the chest-mounted apparatus 106 may be placed on top of electrode pads connected to the monitor 104. In various cases, the chest-mounted apparatus 106 includes an electrically insulative material and/or protection circuitry to protect components of the chest-mounted apparatus 106 when the monitor 104 outputs an electrical signal (e.g., an electrical shock) to the pads. In some examples, one or more of the pads are integrated with the chest-mounted apparatus 106 itself. The chest-mounted apparatus 106, in various cases, includes protection circuitry that prevents the electrical signal from being absorbed (and potentially arming circuitry inside of) the chest-mounted apparatus 106. For instance, the protection circuitry includes one or more diodes that prevents the electrical signal from inducing a current above a threshold level in the circuitry of the chest-mounted apparatus.

In some cases, the housing of the chest-mounted apparatus 106 facilitates the administration of chest compressions to the subject 102. In various examples, an outer surface of the housing includes a material with a relatively high coefficient of friction that prevents slipping between a compressor (e.g., the compressor of a chest compression device or a rescuer's hands) from slipping on the surface of the housing during chest compressions. In some aspects, the housing of the chest-mounted apparatus 106 is printed with icons indicating a target position for the application of chest compressions. For instance, the chest-mounted apparatus 106 may display a graphical icon indicating the midline 112 and/or a center of the chest of the subject 102, when the chest-mounted apparatus 106 is disposed on the skin of the subject 102.

The chest-mounted apparatus 106 includes various sensors configured to detect parameters of the subject 102. Moreover, in some implementations, the chest-mounted apparatus 106 includes at least one processor configured to analyze the detected parameters. For instance, the chest-mounted apparatus 106 may be configured to predict, by analyzing the parameter(s), a physiological condition of the subject 102 and/or a treatment condition of a therapy being administered to the subject 102. As used herein, the term “physiological condition,” and its equivalents, may refer to a state of a subject, such as the subject 102. In some cases, a physiological condition is treatable by a therapy. Examples of physiological conditions determined by the chest-mounted apparatus 106 include pneumothorax, spontaneous circulation, loss of spontaneous circulation, spontaneous respiration, loss of spontaneous respiration, independent ambulation, seizure, and the like. As used herein, the term “treatment condition,” and its equivalents, may refer to a quality or state of a therapy being applied to the subject, such as the subject 102. Various therapies (also referred to as “treatments”), for instance, include chest compressions, assisted ventilation, airway management procedures, electrotherapies (e.g., administration of an electrical shock, administration of pacing pulses, etc.), administration of medication, and the like. Examples of treatment conditions determined by the chest-mounted apparatus 106 include chest compression parameters (e.g., whether chest compressions are applied to the midline 112, chest compression frequency, chest compression depth, etc.), whether a chest compression parameter is within a threshold range, ventilation parameters (e.g., ventilation frequency, ventilation timing, etc.), whether a ventilation parameter is within a threshold range, whether an airway management procedure has been performed correctly, time at which a medication was administered, time at which an administered medication has become effective, and the like.

In various implementations, the chest-mounted apparatus 106 includes multiple accelerometers configured to monitor the acceleration of the chest-mounted apparatus 106 and/or the subject 102 at different positions along the chest of the subject 102. For instance, the chest-mounted apparatus 106 includes a first accelerometer 108 configured to be positioned on a first side of the chest of the subject 102 and a second accelerometer 110 configured to be positioned on a second side of the chest of the subject 102. The first and second sides of the chest of the subject 102, for instance, are divided by a midline 112 of the subject 102.

The positioning of the first accelerometer 108 and the second accelerometer 110 enable detection of physiological conditions associated with differential chest movement. For instance, accelerations detected by the first accelerometer 108 and the second accelerometer 110 can be utilized to infer whether the subject 102 is experiencing a pneumothorax. The subject 102 exhibits a pneumothorax (also referred to as a “collapsed lung”) if air leaks into a space in a pleural space of the subject 102. The pleural space is disposed between each lung and chest wall of the subject 102. The air in the pleural space reduces lung function, which can result in decreased circulating oxygen throughout the body of the subject 102, low blood pressure, and other serious complications. A pneumothorax can occur spontaneously or as a result of one or more causes, such as physical trauma, damage to surrounding tissue, complications from medical interventions, preexisting lung conditions (e.g., tuberculosis, lung disease, asthma, etc.), or any combinations thereof.

In many cases, a pneumothorax impacts a single lung of the subject 102. For instance, the subject 102 exhibits sudden shortness of breath and/or chest pain on one side of the midline 112. Moreover, the subject 102 may exhibit less chest movement during respiration on the side of the pneumothorax compared to the side without the pneumothorax. Accordingly, in various cases, a pneumothorax can be identified, at least in part, based on a discrepancy between an acceleration detected by the first accelerometer 108 and an acceleration detected by the second accelerometer 110 during one or more respiration (or ventilation) cycles of the subject 102. In various cases, a maximum acceleration on one side of the midline 112 that is different than a maximum acceleration on another side of the midline 112 during a respiration cycle is indicative of the pneumothorax. The chest-mounted apparatus 106, in some cases, performs this determination.

In various cases, the acceleration detected by the first accelerometer 108 and/or the acceleration detected by the second accelerometer 110 can be utilized to detect other physiological conditions. For instance, the acceleration(s) are indicative of chest movement due to spontaneous respiration by the subject 102. In various cases, the acceleration(s) can be analyzed in order to determine a rate of spontaneous breathing of the subject 102, or whether the subject 102 is spontaneously breathing. In some implementations, the chest-mounted apparatus 106 infers whether the subject 102 is experiencing a seizure and/or spontaneous circulation upon detecting a predetermined pattern of the acceleration(s). For instance, the chest-mounted apparatus 106 may be configured to perform ballistocardiography using the acceleration(s). In some implementations, the acceleration(s) can be used by the chest-mounted apparatus 106 to determine whether the subject 102 is independently ambulating (e.g., moving independently) or whether the subject 102 is unconscious.

Moreover, the acceleration detected by the first accelerometer 108 and/or the acceleration detected by the second accelerometer 110 can be utilized to detect treatment conditions of the subject 102. In various cases, a rescuer may be performing an airway management procedure on the subject 102. In various cases, the subject 102 may be in respiratory distress due to the presence of an airway obstruction, traumatic injury, or the like. To provide assisted ventilation to the subject 102, the rescuer may at least attempt to insert an endotracheal (ET) tube, supraglottic airway, or other airway adapter, into the airway of the subject 102. The airway adapter may open up a fluidic circuit between the lungs of the subject 102 and an external environment. If appropriately placed, the airway adapter can be physically coupled to a ventilation device, such as a manual ventilation bag or ventilator, that can provide assisted ventilation to the subject 102.

In real-world scenarios, however, it may be difficult for the rescuer to correctly place the airway adapter. For instance, it may be possible for the rescuer to accidentally insert an ET tube or other airway adapter into the esophagus of the subject 102, rather than the trachea of the subject 102. In some cases, even if the airway adapter is placed within the airway, the rescuer may accidentally insert the airway adapter too far into the airway of the subject 102 (which can result, e.g., in physical damage to the subject 102) or too shallowly in the airway of the subject 102 (which can result, e.g., in ineffective ventilation). In various cases, the airway adapter may be inserted so deep into the chest of the subject 102 that the airway adapter enters the right or left main bronchus of the subject 102, which can result in selective ventilation of a single lung.

In various cases, the chest-mounted apparatus 106 is configured to infer whether the airway adapter is placed correctly or incorrectly. In particular examples, the first accelerometer 108 and/or second accelerometer 110 are configured to detect at least one acceleration indicative of the location of the airway adapter in the chest of the subject 102. The chest-mounted apparatus 106, for instance, may infer that the airway adapter has entered the right or left main bronchus of the subject 102 by identifying a discrepancy between the acceleration detected by the first accelerometer 108 and the acceleration detected by the second accelerometer 110 during one or more ventilation cycles of the subject 102.

In some implementations, chest compressions are being applied to the subject 102. In many cases, chest compressions are ideally applied along the midline 112 of the subject 102. If chest compressions are applied away from the midline 112, the chest compressions may be less effective at circulating blood through the body of the subject 102. In various cases, the accelerations detected by the first accelerometer 108 and the second accelerometer 110 can be utilized to infer whether the chest compressions are applied at an ideal position on the chest of the subject 102. For instance, if the acceleration detected by the first accelerometer 108 is consistently greater or lower than the acceleration detected by the second accelerometer 110 during the application of one or more chest compressions, the chest-mounted apparatus 106 may infer that the chest compressions are not being applied to the center of the chest of the subject 102.

In various cases, the chest-mounted apparatus 106 can further detect other treatment conditions based on the acceleration detected by the first accelerometer 108 and the acceleration detected by the second accelerometer 110. For instance, the chest-mounted apparatus 106 may analyze the acceleration(s) to determine a frequency and/or depth of chest compressions applied to the subject 102. In some examples, the acceleration(s) can be utilized to infer whether manual chest compressions are applied to the chest of the subject 102 with full recoil. In some aspects, the acceleration(s) are indicative of assisted ventilation performed on the subject 102. For example, the chest-mounted apparatus 106 may determine a rate and/or insufflation volume of the assisted ventilation based on the acceleration(s). In some cases, the acceleration(s) are indicative of a medication administered to the subject 102. For instance, the acceleration(s) may be indicative of a medication becoming effective at treating a seizure or other condition of the subject 102.

According to various implementations, the chest-mounted apparatus 106 can be equipped with additional sensors to enable more accurate inferences. For instance, the chest-mounted apparatus 106 may include an oximetry sensor 114 configured to detect a blood oxygenation of the subject 102. Although the oximetry sensor 114 is illustrated in FIG. 1 as being integrated with the chest-mounted apparatus 106, implementations are not so limited. In some examples, the oximetry sensor 114 is communicatively coupled with the chest-mounted apparatus 106 and configured to detect a blood oxygenation of the subject 102 at a different location, such as a finger or limb of the subject 102.

The oximetry sensor 114 includes at least one light source and at least one light detector. The light source(s) are configured to emit light having at least one wavelength in a red spectrum and/or an infrared spectrum. For instance, the light source(s) emit light having a wavelength of 660 nm and light having a wavelength of 940 nm. In some examples, the light source(s) include one or more light-emitting diodes (LEDs). In various cases, the light source(s) of the oximetry sensor 114 emit light into the subject 102. The light detector(s) are configured to detect a portion of the emitted light that is transmitted and/or scattered by blood in the body of the subject 102. Due to the differential absorption spectra of hemoglobin and hemoglobin bound to oxygen in the blood, the light detected by the light detector(s) of the oximetry sensor 114 is indicative of a blood oxygenation of the subject 102. In some aspects, the chest-mounted apparatus 106 reports the detected blood oxygenation of the subject 102.

In particular examples, the blood oxygenation detected by the oximetry sensor 114 is used by the chest-mounted apparatus 106 to detect a physiological condition and/or a treatment condition of the subject 102. In some cases, the blood oxygenation improves an inference made, at least in part, based on the accelerations detected by the first accelerometer 108 and the second accelerometer 110. For instance, the chest-mounted apparatus 106 may confirm the presence of the pneumothorax, the improperly placed airway adapter, ineffective ventilation, lack of spontaneous circulation, lack of spontaneous respiration, or the improperly placed chest compressions further in response to determining that the detected blood oxygenation is below a threshold.

According to some examples, the chest-mounted apparatus 106 is configured to detect whether the heart of the subject 102 is spontaneously circulating blood throughout the body of the subject 102. In various cases, oximetry sensor 114 is configured to detect a photoplethysmograph of the subject 102. In some implementations, the chest-mounted apparatus 106 detects peaks in the photoplethysmograph. If the chest-mounted apparatus 106 determines that the peaks in the photplethysmograph are substantially aligned with the chest compressions applied to the subject 102 (e.g., peaks in the acceleration(s) detected by the first accelerometer 108 and/or second accelerometer 110), the chest-mounted apparatus 106 may infer that the subject 102 lacks spontaneous circulation. However, if the chest-mounted apparatus 106 detects one or more peaks in the photoplethysmography that are misaligned with the chest compressions, the chest-mounted apparatus 106 may infer that the heart of the subject 102 is spontaneously circulating blood. In various cases, the chest-mounted apparatus 106 may cause a user to refrain from continuing to administer chest compressions to the subject 102 in response to detecting the spontaneous circulation.

In some implementations, the chest-mounted apparatus 106 includes an additional one or more sensors 116. In various implementations, the sensor(s) 116 include one or more physiological sensors, such as any of the physiological sensors described within this disclosure. In some examples, the sensor(s) 116 include one or more other types of sensors, such as accelerometers, microphones, transducers, light sensors, pressure sensors, or the like. The sensor(s) 116 are configured to detect one or more parameters of the subject 102 or the chest-mounted apparatus 106 itself. In various implementations, the chest-mounted apparatus 106 is configured to detect a physiological condition of the subject 102, a treatment condition of the subject 102, a condition of the chest-mounted apparatus 106, or a combination thereof, based on the parameter(s) detected by the sensor(s) 116, the acceleration detected by the first accelerometer 108, the acceleration detected by the second accelerometer 110, the blood oxygenation detected by the oximetry sensor 114, or any combination thereof.

In particular examples, a pressure sensor among the sensor(s) 116 can be utilized to determine whether manual chest compressions are being applied to the chest of the subject 102 with full recoil. In ideal circumstances, a rescuers hands should be raised from the chest of the subject between chest compressions. In various implementations, the pressure sensor is configured to detect whether an applied pressure to the chest of the subject 102 lowers to less than a threshold pressure between chest compressions. In some cases, the chest compressions are detected based on the pressure and/or acceleration(s) detected by the first accelerometer 108 and/or the second accelerometer 110. In various cases, the chest-mounted apparatus 106 determines whether full recoil has been achieved between consecutive chest compressions applied to the chest of the subject 102.

In some implementations, one or more microphones or piezoelectric transducers among the sensor(s) 116 are utilized by the chest-mounted apparatus to determine a physiological condition of the subject 102. For instance, the sensor(s) 116 include a membrane disposed between the skin of the subject 102 and a container of air, wherein a pressure within the container of air is indicative of sound emitted by the subject 102. The microphone(s) and/or transducer(s), for instance, are configured to detect sound from the body of the subject 102. The sound, for instance, is indicative of heart contraction, blood flow, air in the airway of the subject 102, or any combination thereof. In various implementations, the sound detected by the sensor(s) 116 enables the chest-mounted apparatus to identify and/or confirm that the heart of the subject 102 is spontaneously pumping blood, that the subject 102 is spontaneously breathing, or the like.

According to some examples, the chest-mounted apparatus 106 is configured to a condition of the chest-mounted apparatus 106 itself based on the acceleration detected by the first accelerometer 108, the acceleration detected by the second accelerometer 110, the parameter(s) detected by the sensor(s) 116, or any combination thereof. Examples of device conditions include a temperature of the chest-mounted apparatus 106, an ambient temperature, an acceleration of the chest-mounted apparatus 106, a battery state (e.g., charge level of a battery of the chest-mounted apparatus 106), or the like.

In some aspects, one or more parameter(s) detected by the chest-mounted apparatus 106 can be utilized for gating. For instance, the chest-mounted apparatus 106 may deactivate, or discard measurements from, the oximetry sensor 114 and/or the sensor(s) 116 upon detecting that the acceleration detected by the first accelerometer 108 and/or the acceleration detected by the second accelerometer 110 is above a threshold.

In some implementations, the chest-mounted apparatus 106 outputs an indication of a parameter, a physiological condition of the subject 102, a treatment condition of the subject 102, a device condition of the chest-mounted apparatus 106, or any combination thereof. For example, the chest-mounted apparatus 106 includes an output device, such as a light source, a display, a speaker, a haptic feedback device, or the like. The output device, in various cases, may output a signal (e.g., a visual signal, an audible signal, a vibration signal, or the like) indicative of the parameter, the physiological condition, the treatment condition, the device condition, or a combination thereof.

Although FIG. 1 has primarily been described with reference to the chest-mounted apparatus 106 performing various analyses, implementations are not so limited. For instance, the chest-mounted apparatus 106 may be configured to communicate, to the monitor 104, one or more of the parameters detected by the chest-mounted apparatus 106. The monitor 104, in some cases, determines a condition of the chest-mounted apparatus 106, a treatment applied to the subject 102, or a physiological condition of the subject 102, based on the parameter(s) detected and communicated by the chest-mounted apparatus 106.

In various cases, the chest-mounted apparatus 106 communicates with the monitor 104 by exchanging one or more communication signal(s) 118 over at least one wireless interface. Examples of the wireless interface include, for instance, a BLUETOOTH™ interface, a Near-Field Communication (NFC) interface, a WI-FI™ interface, or any other interface utilizing any other wireless communication protocol described herein. For instance, the communication signal(s) 118 include electromagnetic signals. In some implementations, the communication signal(s) 118 include ultrasonic signals that encode various data described herein.

According to various implementations, the communication signal(s) 118 include one or more indications of parameter(s) detected by the chest-mounted apparatus 106. In various cases, the communication signal(s) 118 further include one or more indications of times at which the parameters were detected by the chest-mounted apparatus 106. For instance, the communication signal(s) 118 indicate one or more timestamps corresponding to the times at which the parameter(s) were detected by the chest-mounted apparatus 106.

The communication signal(s) 118 encode one or more data packets, for instance. In various implementations, each data packet is composed of a header (e.g., indicating source and destination addresses) and a payload containing substantive data, such as indications of the parameter(s) and/or the timestamps. According to some cases, an individual data packet within the communication signal(s) 118 indicates more than one type of parameter measurement. For instance, the data packet may include both an indication of an acceleration detected by the first accelerometer 108 and a blood oxygenation detected by the oximetry sensor 114. In various implementations, the data indicating the different parameters are combined in the payload of the data packet. For instance, the chest-mounted apparatus 106 may be configured to interleave the data indicating one parameter (e.g., the acceleration) with data indicating another parameter (e.g., the blood oxygenation). In some instances, the interleaved data represents parameter measurements detected at the same time. For instance, the data packet may also include an indication at which the parameters were detected. By interleaving the measurements in the data packets, the chest-mounted apparatus 106 can efficiently report multiple types of measurements to the monitor 104 substantially in real time. Moreover, the parameters may be inherently time-aligned within the data packet (or each of a stream of multiple data packets), enhancing analyses by the monitor 104 that depend on patterns across multiple parameters (e.g., using a photoplethysmography and acceleration to detect a return of spontaneous circulation during chest compressions). According to various implementations, the monitor 104 may be configured to determine a physiological condition of the subject 102, a treatment condition of the subject 102, or a device condition of the chest-mounted apparatus 106 based on the communication signal(s) 118.

In some examples, the communication signal(s) 118 include one or more indications of conditions determined by the chest-mounted apparatus 106. For instance, in some cases, the communication signal(s) 118 include data packets encoding indications of one or more conditions determined by the chest-mounted apparatus 106. The conditions, for instance, may include any of the physiological conditions, treatment conditions, or device conditions described herein.

In some implementations, the monitor 104 is configured to communicate one or more parameters detected by the monitor 104 to the chest-mounted apparatus 106 via the communication signal(s) 118. For example, the monitor 104 may detect one or more physiological parameters of the subject 102 via one or more sensors. The monitor 104 may package indication(s) of the parameter(s) in data packets within the communication signal(s) 118. When the chest-mounted apparatus 106 receives the indication(s) of the parameter(s), the chest-mounted apparatus 106 may use the indication(s) to determine a physiological condition of the subject 102, a treatment condition of the subject 102, or a device condition of the chest-mounted apparatus 106.

The communication signal(s) 118, in various cases, may include encrypted data. For instance, the chest-mounted apparatus 106 may encrypt data indicative of parameter(s) and/or condition(s) prior to encoding the data in the communication signal(s) 118. As used herein, the term “encrypt,” and its equivalents, refers to a process of translating data from one format (e.g., an unencoded format) into an encoded format. In various cases, the encoded format is referred to as “ciphertext.” Unencoded data, which has not been encrypted, may be referred to as being in “plaintext.” In various examples, the chest-mounted apparatus 106 encrypts data using at least one encryption key. An encryption key is a parameter that defines the translation of data from the one format into the encoded format. As used herein, the term “decrypt,” and its equivalents, refers to a process of translating data from an encoded format into another format (e.g., an unencoded format), such as a plaintext format. In various cases, the monitor 104 is configured to convey information to the chest-mounted apparatus 106 (e.g., via the communication signal(s) 118) that enables the chest-mounted apparatus 106 to extract and/or generate an encryption key. The chest-mounted apparatus 106 encrypts data transmitted to the monitor 104 (via the communication signal(s) 118) using the encryption key. The monitor 104, in turn, is configured to decrypt the data it receives via the communication signal(s) 118.

Various cryptographic techniques can be utilized in accordance with the features described in this disclosure. For example, data can be encrypted and decrypted via a symmetric key, wherein the encryption key and the decryption key are equivalent. In some cases, data can be encrypted and decrypted via asymmetric keys, wherein the encryption key and the decryption key are different. Cryptographic hash functions (CHFs) are examples of cryptographic techniques. Examples of cryptographic techniques include the Data Encryption Standard (DES), Advanced Encryption Standard (AES), Elliptic Curve Cryptography (ECC), Rivest-Shamir-Adleman (RSA), Secure Hash Algorithm (SHA)-1, SHA-2, SHA-3, BLAKE, BLAKE2, BLAKE3, WHIRLPOOL, MD2, MD4, MD5, MD6, Temporal Key Integrity Protocol (TKIP), Rivest cipher 4 (RC4), variably modified permutation composition (VMPC), blowfish, Twofish, Threefish, Tiny Encryption Algorithm (TEA), Extended TEA (XTEA), Corrected Block TEA (XXTEA), Diffie-Hellman exchange (DHE), elliptic curve DHE, super-singular isogeny Diffie-Hellman (SIDH) key exchange, and so on. Any suitable encryption or decryption technique can be used in accordance with implementations of this disclosure. By encrypting the data in the communication signal(s) 118, exposure of sensitive, private medical information of the subject 102 can be avoided.

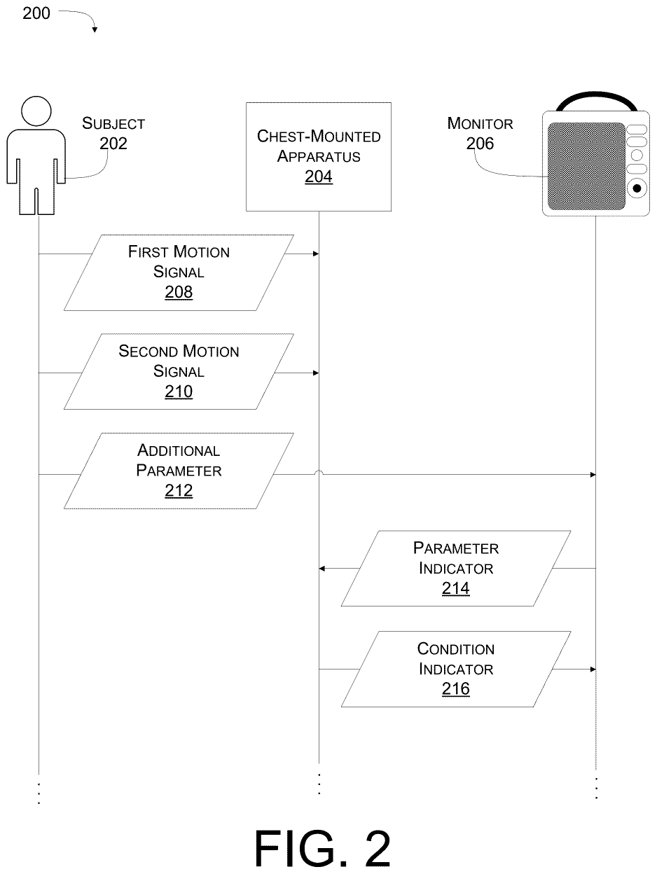

FIG. 2 illustrates example signaling 200 for determining a condition. The signaling 200 is between a subject 202, a chest-mounted apparatus 204, and a monitor 206. In some examples, the subject 202 is the subject 102, the chest-mounted apparatus 204 is the chest-mounted apparatus 106, the monitor 206 is the monitor 104, or any combination thereof.

The chest-mounted apparatus 204 detects a first motion signal 208 from the subject 202. In various cases, the first motion signal 208 is associated with a first side of the subject 202. For instance, a first accelerometer or first gyroscope of the chest-mounted apparatus 204 is configured to detect the first motion signal 208.

The chest-mounted apparatus 204 further detects a second motion signal 210 from the subject 202. The second motion signal 210, for instance, is associated with a second side of the subject 202. In various cases, the first side and the second side of the subject 202 are separated by a midline of the subject 202. For example, a second accelerometer or second gyroscope of the chest-mounted apparatus 204 is configured to detect the second motion signal 210. In some cases, the chest-mounted apparatus 204 detects the first motion signal 208 and the second motion signal 210 at the same time.

The monitor 206 detects an additional parameter 212 from the subject 202. In various examples, the additional parameter 212 is a physiological parameter. In some cases, the additional parameter 212 is a non-physiological parameter associated with the subject 202. The monitor 206, for instance, includes a sensor configured to detect the additional parameter 212 from the subject 202. In some examples, the additional parameter 212 is detected at the same time as the first motion signal 208 and/or the second motion signal 210.

The monitor 206 transmits a parameter indicator 214 to the chest-mounted apparatus 204. The parameter indicator 214 provides the parameter detected by the monitor 206 to the chest-mounted apparatus 204. In some implementations, the parameter indicator 214 is transmitted wirelessly to the chest-mounted apparatus 204 over a wireless interface. For example, the parameter indicator 214 is encoded in a data packet that is included in a communication signal transmitted by the monitor 206 to the chest-mounted apparatus 204. In some aspects, the parameter indicator 214 includes an indication of the time at which the additional parameter 212 was detected.

In various implementations, the chest-mounted apparatus 204 determines a condition based on the first motion signal 208, the second motion signal 210, and the parameter indicator 214. In some cases, the chest-mounted apparatus 204 determines a physiological condition, such as a presence or absence of a pneumothorax, a presence or absence of spontaneous circulation, a presence or absence of spontaneous breathing, a presence or absence of a seizure, or any combination thereof. In some examples, the chest-mounted apparatus 204 determines a treatment condition, such as a condition associated with chest compressions applied to the subject 102, assisted ventilation applied to the subject 102, an electrotherapy applied to the subject 102, a medication administered to the subject, or a combination thereof.

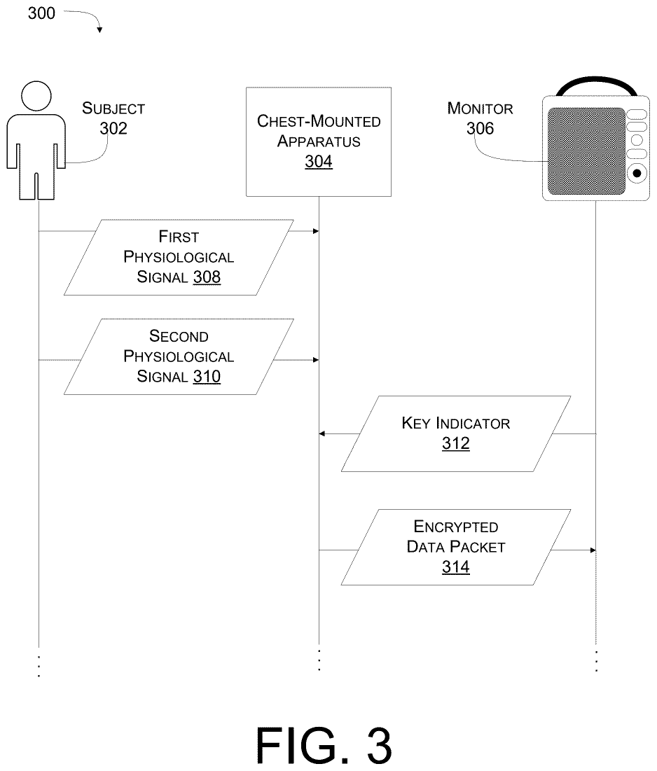

FIG. 3 illustrates example signaling 300 for reporting one or more parameters. The signaling 300 is between a subject 302, a chest-mounted apparatus 304, and a monitor 306. In some examples, the subject 302 is the subject 102, the chest-mounted apparatus 304 is the chest-mounted apparatus 106, the monitor 306 is the monitor 104, or any combination thereof.

The chest-mounted apparatus 304 detects a first physiological signal 308 from the subject 302. For instance, the chest-mounted apparatus 304 includes a first sensor configured to detect a first parameter of the subject 302 based on the first physiological signal 308. In some cases, the first physiological signal 308 includes an electrical signal, an acceleration, an optical transmittance, a pressure, a gas or other substance, a temperature, a sound, a light signal, or the like.

The chest-mounted apparatus 304 detects a second physiological signal 310 from the subject 302. For instance, the chest-mounted apparatus 304 includes a second sensor configured to detect a second parameter of the subject 302 based on the first physiological signal 308. In some cases, the second physiological signal 310 includes an electrical signal, an acceleration, an optical transmittance, a pressure, a gas or other substance, a temperature, a sound, a light signal, or the like. The first physiological signal 308 may be a different type of signal than the second physiological signal 310. In some implementations, the chest-mounted apparatus 304 detects the first physiological signal 308 and the second physiological signal 310 at the same time.

The chest-mounted apparatus 304 is configured to generate a raw data packet based on the first parameter and the second parameter. For example, a payload of the raw data packet includes data indicating the first parameter and data indicating the second parameter. In various cases, the payload of the raw data packet further includes an indication of the time(s) at which the first parameter and the second parameter were detected. In various implementations, the data indicating the first parameter and the data indicating the second parameter are interleaved in the raw data packet.

The chest-mounted apparatus 304 receives, from the monitor 306, a key indicator 312. In various implementations, the chest-mounted apparatus 304 generates or extracts an encryption key based on the key indicator 312. In various cases, the chest-mounted apparatus 304 generates an encrypted data packet 314 by encrypting the raw data packet. The chest-mounted apparatus 304 sends the encrypted data packet 314 to the monitor 306. In various cases, the monitor 306 extracts the first and second parameters of the subject 302 by decrypting the encrypted data packet 314.

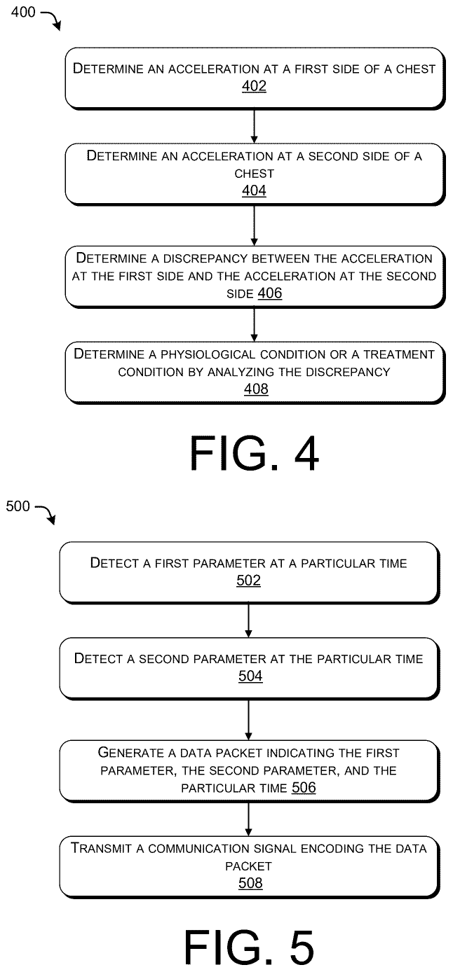

FIG. 4 illustrates an example process 400 for determining a condition of a subject. The process 400 is performed by an entity, such as a chest-mounted apparatus (e.g., the chest-mounted apparatus 106, the chest-mounted apparatus 204, the chest-mounted apparatus 304, or a combination thereof), a monitor (e.g., the monitor 104, the monitor 206, the monitor 306, or a combination thereof), a medical device, at least one processor, a computing device, or a combination thereof.

At 402, the entity determines an acceleration at a first side of a chest of the subject. In various cases, the acceleration is detected by a first accelerometer disposed on the first side of the chest. For instance, the first accelerometer is part of a chest-mounted apparatus disposed on the chest of the subject. In some implementations, the acceleration at the first side is detected during a respiration cycle, a ventilation cycle, or a chest compression cycle of the subject.

At 404, the entity determines an acceleration at a second side of the chest. In various cases, the acceleration is detected by a second accelerometer disposed on the second side of the chest. For instance, the second accelerometer is part of a chest-mounted apparatus disposed on the chest of the subject. The first side and the second side of the chest, for instance, are divided by a midline of the subject. In some implementations, the acceleration at the second side is detected during a respiration cycle, a ventilation cycle, or a chest compression cycle of the subject. For instance, the acceleration at the first side and the acceleration at the second side are detected at substantially the same time.

At 406, the entity determines a discrepancy between the acceleration at the first side and the acceleration at the second side. For instance, the entity subtracts a maximum acceleration at the first side during a time period from a maximum acceleration at the second side during the time period. In some implementations, the entity generates a waveform representing an absolute value of a difference between the acceleration at the first side and the acceleration at the second side, wherein each data point of the waveform represents a difference between acceleration measurements at the first side and second side taken at the same time.

At 408, the entity determines a physiological condition or a treatment condition by analyzing the discrepancy. In some implementations, the physiological condition includes a pneumothorax. For example, if the discrepancy is greater than a threshold for one or more respiration or ventilation cycles, the entity may infer that the subject has a pneumothorax. In some cases, the treatment condition includes whether an endotracheal tube (or other airway adapter) is properly placed in the airway of the subject. For example, the entity may infer that the endotracheal tube is improperly placed by determining that the discrepancy is greater than a threshold for one or more ventilation cycles of the subject. According to some implementations, the entity is configured to infer an insufflation volume applied to the subject based on the acceleration at the first side and the acceleration at the second side. A lack of chest movement may indicate a lack of air entering the lungs. In various cases, integrating a waveform of the acceleration of the chest with respect to time can produce a waveform of the velocity of the chest with respect to time, and integrating the waveform of the velocity of the chest with respect to time can produce a waveform of the position of the chest with respect to time. In various cases, the insufflation volume can be estimated based on the waveform of the position of the chest with respect to time. In some implementations, the insufflation volume is further based on other factors, such as a size of the subject (e.g., small, medium, or large) in height, a chest circumference of the subject, a chest width of the subject, or the like. In some aspects, the entity estimates the insufflation volume based on a prestored lookup table that correlates chest position and another factor (e.g., subject size, chest circumference, chest width, etc.) with empirically derived insufflation volumes.

In some cases, the entity further determines whether the insufflation volume is within a threshold range. According to some examples, the entity determines a parameter of chest compressions applied to the subject. For example, the discrepancy may be indicative of a position at which the chest compressions are applied, wherein a greater discrepancy may be indicative of chest compressions applied farther from the midline of the subject. In some cases, the entity determines a depth of the chest compressions based on the acceleration at the first side and/or the acceleration at the second side.

Optionally, the entity determines the physiological condition or treatment condition based on one or more additional parameters. In some implementations, the entity detects an additional parameter (e.g., a physiological parameter of the subject) and determines the physiological condition or treatment condition based on at least one of the accelerations and the additional parameter. For instance, the entity may confirm the pneumothorax, the improperly placed airway adapter, an insufficient insufflation volume, or chest compressions applied away from the center of the chest by determining that a blood oxygenation and/or EtCO₂ of the subject is below a threshold. In some cases, the entity receives an indication of an acceleration of a medical device coupled with the subject. For instance, the entity receives, from a monitor, a communication signal indicating an acceleration of the monitor. In various implementations, the entity may determine the physiological condition or the treatment condition based on the acceleration of the monitor. For instance, the entity may infer that the subject is independently ambulating in response to determining that a discrepancy between the acceleration of the medical device and the acceleration at the first side of the chest (or the acceleration at the second side of the chest) is above a threshold.

In various cases, the entity may output an indication of the physiological condition and/or treatment condition. For example, the entity may output the indication to a user. In some implementations, the entity transmits a communication signal encoding the indication to an external device. According to some implementations, the entity encrypts the indication within the communication signal.

FIG. 5 illustrates an example process 500 for communicating parameters. The process 500 is performed by an entity, such as a chest-mounted apparatus (e.g., the chest-mounted apparatus 106, the chest-mounted apparatus 204, the chest-mounted apparatus 304, or a combination thereof), a monitor (e.g., the monitor 104, the monitor 206, the monitor 306, or a combination thereof), a medical device, at least one processor, a computing device, or a combination thereof.

At 502, the entity detects a first parameter at a particular time. The first parameter is a parameter of a subject or a device (e.g., a chest-mounted apparatus). In some implementations, the first parameter is an ECG, EEG, EMG, temperature, blood oxygenation, blood pressure, blood flow parameter, airway parameter, sound, or acceleration. In some cases, the first parameter is a physiological parameter of the subject.

At 504, the entity detects a second parameter at the particular time. The second parameter is a parameter of a subject or a device (e.g., the chest-mounted apparatus). In some implementations, the second parameter is an ECG, EEG, EMG, temperature, blood oxygenation, blood pressure, blood flow parameter, airway parameter, sound, or acceleration. In some cases, the second parameter is a physiological parameter of the subject.

According to various cases, the second parameter is a different type of parameter than the first parameter. For instance, in some examples, the first parameter is an acceleration of a chest of the subject, and the second parameter is a blood flow parameter of the subject. The blood flow parameter include any physiological parameter indicative of blood circulation, such as blood oxygenation (e.g., pulse oxygenation), a capnograph, EtCO₂, a blood velocity, a blood flow rate, or the like. Optionally, the entity infers or predicts a condition of the subject or a condition of a treatment applied to the subject based on the first parameter and/or the second parameter. For example, if the entity determines that an acceleration at the particular time is below a threshold and that the blood flow parameter at the particular time is above a threshold, the entity may infer that the subject has spontaneous circulation.

At 506, the entity generates a data packet indicating the first parameter, the second parameter, and the particular time. For instance, the entity interleaves data indicating the first parameter, data indicating the second parameter, and data indicating the particular time, within the payload of the data packet. In some implementations, the data in the payload of the data packet is encrypted. For instance, the entity encrypts the data using an encryption key. In some implementations, the entity derives or extracts the encryption key from a communication signal transmitted from an external device to the entity.

At 508, the entity transmits a communication signal encoding the data packet. The communication signal may include an electromagnetic signal and/or an ultrasonic signal. In various implementations, the communication signal is received by an external medical device, such as an external monitor. In various implementations, the external medical device analyzes and/or outputs data within the data packet. In some cases, the entity transmits a stream of data packets corresponding to parameter measurements detected in substantially real-time. Each data packet within the stream can be generated using similar techniques to those described with respect to the data packet generated at 506.

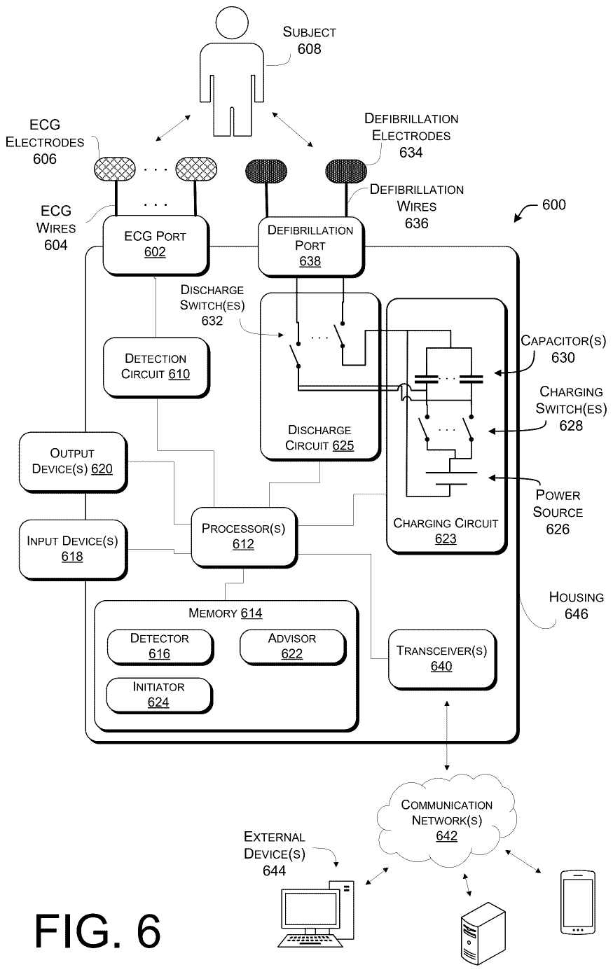

FIG. 6 illustrates an example of an external defibrillator 600 configured to perform various functions described herein. For example, the external defibrillator 600 is the monitor 104 described above with reference to FIG. 1.

The external defibrillator 600 includes an electrocardiogram (ECG) port 602 connected to multiple ECG wires 604. In some cases, the ECG wires 604 are removeable from the ECG port 602. For instance, the ECG wires 604 are plugged into the ECG port 602 via connectors. The ECG wires 604 are connected to ECG electrodes 606, respectively. In various implementations, the ECG electrodes 606 are disposed on different locations on a subject 608. A detection circuit 610 is configured to detect relative voltages between the ECG electrodes 606. These voltages are indicative of the electrical activity of the heart of the subject 608.

In various implementations, the ECG electrodes 606 are in contact with the different locations on the skin of the subject 608. In some examples, a first one of the ECG electrodes 606 is placed on the skin between the heart and right arm of the subject 608, a second one of the ECG electrodes 606 is placed on the skin between the heart and left arm of the subject 608, and a third one of the ECG electrodes 606 is placed on the skin between the heart and a leg (either the left leg or the right leg) of the subject 608. In these examples, the detection circuit 610 is configured to measure the relative voltages between the first, second, and third ECG electrodes 606. Respective pairings of the ECG electrodes 606 are referred to as “leads,” and the voltages between the pairs of ECG electrodes 606 are known as “lead voltages.” In some examples, more than three ECG electrodes 606 are included, such that 5-lead or 12-lead ECG signals are detected by the detection circuit 610.

The detection circuit 610 includes at least one analog circuit, at least one digital circuit, or a combination thereof. The detection circuit 610 receives the analog electrical signals from the ECG electrodes 606, via the ECG port 602 and the ECG wires 604. In some cases, the detection circuit 610 includes one or more analog filters configured to filter noise and/or artifact from the electrical signals. The detection circuit 610 includes an analog-to-digital (ADC) in various examples. The detection circuit 610 generates a digital signal indicative of the analog electrical signals from the ECG electrodes 606. This digital signal can be referred to as an “ECG signal” or an “ECG.”

In some cases, the detection circuit 610 further detects an electrical impedance between at least one pair of the ECG electrodes 606. For example, the detection circuit 610 includes, or otherwise controls, a power source that applies a known voltage (or current) across a pair of the ECG electrodes 606 and detects a resultant current (or voltage) between the pair of the ECG electrodes 606. The impedance is generated based on the applied signal (voltage or current) and the resultant signal (current or voltage). In various cases, the impedance corresponds to respiration of the subject 608, chest compressions performed on the subject 608, and other physiological states of the subject 608. In various examples, the detection circuit 610 includes one or more analog filters configured to filter noise and/or artifact from the resultant signal. The detection circuit 610 generates a digital signal indicative of the impedance using an ADC. This digital signal can be referred to as an “impedance signal” or an “impedance.”

The detection circuit 610 provides the ECG signal and/or the impedance signal one or more processors 612 in the external defibrillator 600. In some implementations, the processor(s) 612 includes a central processing unit (CPU), a graphics processing unit (GPU), both CPU and GPU, or other processing unit or component known in the art.

The processor(s) 612 is operably connected to memory 614. In various implementations, the memory 614 is volatile (such as random access memory (RAM)), non-volatile (such as read only memory (ROM), flash memory, etc.) or some combination of the two. The memory 614 stores instructions that, when executed by the processor(s) 612, causes the processor(s) 612 to perform various operations. In various examples, the memory 614 stores methods, threads, processes, applications, objects, modules, any other sort of executable instruction, or a combination thereof. In some cases, the memory 614 stores files, databases, or a combination thereof. In some examples, the memory 614 includes, but is not limited to, RAM, ROM, electrically erasable programmable read-only memory (EEPROM), flash memory, or any other memory technology. In some examples, the memory 614 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 612 and/or the external defibrillator 600. In some cases, the memory 614 at least temporarily stores the ECG signal and/or the impedance signal.

In various examples, the memory 614 includes a detector 616, which causes the processor(s) 612 to determine, based on the ECG signal and/or the impedance signal, whether the subject 608 is exhibiting a particular heart rhythm. For instance, the processor(s) 612 determines whether the subject 608 is experiencing a shockable rhythm that is treatable by defibrillation. Examples of shockable rhythms include ventricular fibrillation (VF) and ventricular tachycardia (VT). In some examples, the processor(s) 612 determines whether any of a variety of different rhythms (e.g., asystole, sinus rhythm, atrial fibrillation (AF), etc.) are present in the ECG signal.

The processor(s) 612 is operably connected to one or more input devices 618 and one or more output devices 620. Collectively, the input device(s) 618 and the output device(s) 620 function as an interface between a user and the defibrillator 600. The input device(s) 618 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 620 includes at least one of a display, a speaker, a haptic output device, a printer, or any combination thereof. In various examples, the processor(s) 612 causes a display among the input device(s) 618 to visually output a waveform of the ECG signal and/or the impedance signal. In some implementations, the input device(s) 618 includes one or more touch sensors, the output device(s) 620 includes a display screen, and the touch sensor(s) are integrated with the display screen. Thus, in some cases, the external defibrillator 600 includes a touchscreen configured to receive user input signal(s) and visually output physiological parameters, such as the ECG signal and/or the impedance signal.

In various implementations, the input device(s) 618 further include, or are otherwise connected to, one or more physiological sensors. The physiological sensor(s), for instance, are configured to detect one or more physiological parameters of the subject 608. Examples of the physiological sensor(s) include a blood pressure sensor (e.g., a blood pressure cuff, invasive blood pressure sensor, or the like), an airway sensor (e.g., a sensor configured to detect a partial pressure of CO₂ and/or O₂ in an airway of the subject 608), a blood oxygenation sensor (e.g., a pulse oximeter, regional oxygenation sensor, or the like), a thermometer, a pulse sensor, a blood flow sensor (e.g., an ultrasound transducer configured to detect blood flow using Doppler-based techniques), an airway pressure sensor, or any combination thereof. The input device(s) 618, in some cases, includes one or more sensors configured to detect other characteristics of the subject 608. For example, the input device(s) 618 includes an accelerometer, gyroscope, microphone, or any combination thereof. In various implementations, the processor(s) 612 is configured to assess a condition of the subject 608 by analyzing data derived from signals detected by the input device(s) 618.

In some examples, the memory 614 includes an advisor 622, which, when executed by the processor(s) 612, causes the processor(s) 612 to generate advice and/or control the output device(s) 620 to output the advice to a user (e.g., a rescuer). In some examples, the processor(s) 612 provides, or causes the output device(s) 620 to provide, an instruction to perform CPR on the subject 608. In some cases, the processor(s) 612 evaluates, based on the ECG signal, the impedance signal, or other physiological parameters, CPR being performed on the subject 608 and causes the output device(s) 620 to provide feedback about the CPR in the instruction. According to some examples, the processor(s) 612, upon identifying that a shockable rhythm is present in the ECG signal, causes the output device(s) 620 to output an instruction and/or recommendation to administer a defibrillation shock to the subject 608.

The memory 614 also includes an initiator 624 which, when executed by the processor(s) 612, causes the processor(s) 612 to control other elements of the external defibrillator 600 in order to administer a defibrillation shock to the subject 608. In some examples, the processor(s) 612 executing the initiator 624 selectively causes the administration of the defibrillation shock based on determining that the subject 608 is exhibiting the shockable rhythm and/or based on an input from a user (received, e.g., by the input device(s) 618. In some cases, the processor(s) 612 causes the defibrillation shock to be output at a particular time, which is determined by the processor(s) 612 based on the ECG signal and/or the impedance signal.

The processor(s) 612 is operably connected to a charging circuit 623 and a discharge circuit 625. In various implementations, the charging circuit 623 includes a power source 626, one or more charging switches 628, and one or more capacitors 630. The power source 626 includes, for instance, a battery. The processor(s) 612 initiates a defibrillation shock by causing the power source 626 to charge at least one capacitor among the capacitor(s) 630. For example, the processor(s) 612 activates at least one of the charging switch(es) 628 in the charging circuit 623 to complete a first circuit connecting the power source 626 and the capacitor to be charged. Then, the processor(s) 612 causes the discharge circuit 625 to discharge energy stored in the charged capacitor across a pair of defibrillation electrodes 634, which are in contact with the subject 608. For example, the processor(s) 612 deactivates the charging switch(es) 628 completing the first circuit between the capacitor(s) 630 and the power source 626, and activates one or more discharge switches 632 completing a second circuit connecting the charged capacitor 630 and at least a portion of the subject 608 disposed between defibrillation electrodes 634.

The energy is discharged from the defibrillation electrodes 634 in the form of a defibrillation shock. For example, the defibrillation electrodes 634 are connected to the skin of the subject 608 and located at positions on different sides of the heart of the subject 608, such that the defibrillation shock is applied across the heart of the subject 608. The defibrillation shock, in various examples, depolarizes a significant number of heart cells in a short amount of time. The defibrillation shock, for example, interrupts the propagation of the shockable rhythm (e.g., VF or VT) through the heart. In some examples, the defibrillation shock is 200 J or greater with a duration of about 0.015 seconds. In some cases, the defibrillation shock has a multiphasic (e.g., biphasic) waveform. The discharge switch(es) 632 are controlled by the processor(s) 612, for example. In various implementations, the defibrillation electrodes 634 are connected to defibrillation wires 636. The defibrillation wires 636 are connected to a defibrillation port 638, in implementations. According to various examples, the defibrillation wires 636 are removable from the defibrillation port 638. For example, the defibrillation wires 636 are plugged into the defibrillation port 638.

In various implementations, the processor(s) 612 is operably connected to one or more transceivers 640 that transmit and/or receive data over one or more communication networks 642. For example, the transceiver(s) 640 includes a network interface card (NIC), a network adapter, a local area network (LAN) adapter, or a physical, virtual, or logical address to connect to the various external devices and/or systems. In various examples, the transceiver(s) 640 includes any sort of wireless transceivers capable of engaging in wireless communication (e.g., radio frequency (RF) communication). For example, the communication network(s) 642 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 640 includes other wireless modems, such as a modem for engaging in WI-FI®, WIGIG®, WIMAX®, BLUETOOTH®, or infrared communication over the communication network(s) 642. In some implementations, the transceiver(s) 640 is configured to perform wireless communication using an ultrasonic carrier. For instance, the transceiver(s) 640 is configured to emit an ultrasonic signal encoding data over the communication network(s) 642.

The defibrillator 600 is configured to transmit and/or receive data (e.g., ECG data, impedance data, data indicative of one or more detected heart rhythms of the subject 608, data indicative of one or more defibrillation shocks administered to the subject 608, etc.) with one or more external devices 644 via the communication network(s) 642. The external devices 644 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 642. In some examples, the external device(s) 644 is located remotely from the defibrillator 600, such as at a remote clinical environment (e.g., a hospital). According to various implementations, the processor(s) 612 causes the transceiver(s) 640 to transmit data to the external device(s) 644. In some cases, the transceiver(s) 640 receives data from the external device(s) 644 and the transceiver(s) 640 provide the received data to the processor(s) 612 for further analysis.

In various implementations, the external defibrillator 600 also includes a housing 646 that at least partially encloses other elements of the external defibrillator 600. For example, the housing 646 encloses the detection circuit 610, the processor(s) 612, the memory 614, the charging circuit 623, the transceiver(s) 640, or any combination thereof. In some cases, the input device(s) 618 and output device(s) 620 extend from an interior space at least partially surrounded by the housing 646 through a wall of the housing 646. In various examples, the housing 646 acts as a barrier to moisture, electrical interference, and/or dust, thereby protecting various components in the external defibrillator 600 from damage.

In some implementations, the external defibrillator 600 is an automated external defibrillator (AED) operated by an untrained user (e.g., a bystander, layperson, etc.) and can be operated in an automatic mode. In automatic mode, the processor(s) 612 automatically identifies a rhythm in the ECG signal, makes a decision whether to administer a defibrillation shock, charges the capacitor(s) 630, discharges the capacitor(s) 630, or any combination thereof. In some cases, the processor(s) 612 controls the output device(s) 620 to output (e.g., display) a simplified user interface to the untrained user. For example, the processor(s) 612 refrains from causing the output device(s) 620 to display a waveform of the ECG signal and/or the impedance signal to the untrained user, in order to simplify operation of the external defibrillator 600.

In some examples, the external defibrillator 600 is a monitor-defibrillator utilized by a trained user (e.g., a clinician, an emergency responder, etc.) and can be operated in a manual mode or the automatic mode. When the external defibrillator 600 operates in manual mode, the processor(s) 612 cause the output device(s) 620 to display a variety of information that may be relevant to the trained user, such as waveforms indicating the ECG data and/or impedance data, notifications about detected heart rhythms, and the like.

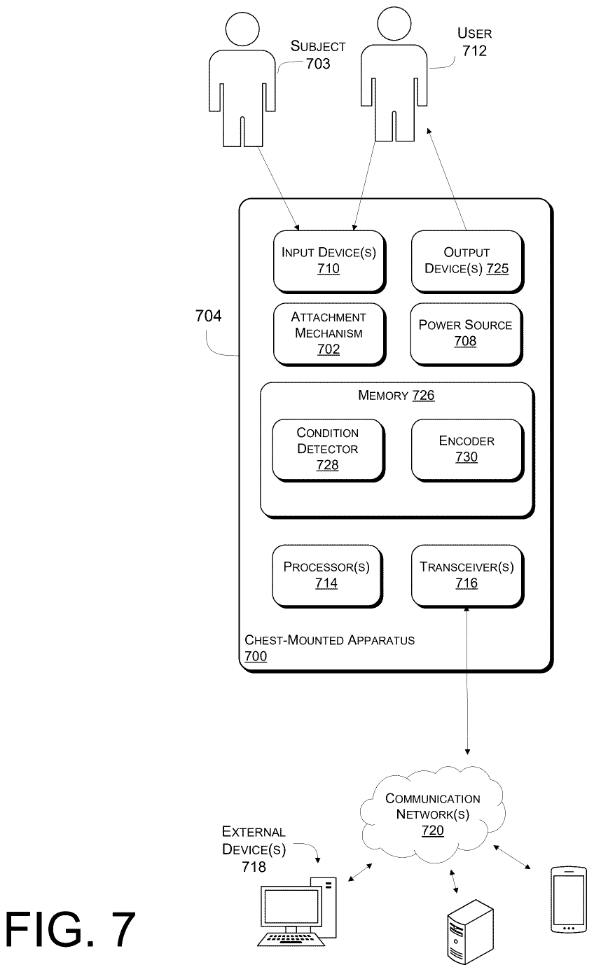

FIG. 7 illustrates a chest-mounted apparatus 700 configured to perform various functions described herein. For example, the chest-mounted apparatus 700 is the chest-mounted apparatus 106 described above with reference to FIG. 1.

In various implementations, the chest-mounted apparatus 700 includes an attachment mechanism 702 configured to attach a housing 704 of the chest-mounted apparatus 700 onto skin of a subject 703. In some implementations, the attachment mechanism 702 includes a biocompatible adhesive. In some aspects, the biocompatible adhesive serves as a matching layer with an intermediate acoustic attenuation between the acoustic attenuation of the housing 704 and the acoustic attenuation of the skin of the subject 703. Accordingly, the biocompatible adhesive may facilitate the transmission of ultrasound. In some implementations, the biocompatible adhesive is optically transparent, such that it can substantially transmit light between the body of the subject 703 and the chest-mounted apparatus 700. According to some cases, the attachment mechanism 702 includes at least one strap, at least one band, or the like, configured to wrap around a portion of the body of the subject 703. In various cases, the attachment mechanism 702 includes a buckle, an elastic material, a hook-and-loop fastener material, or the like. In various cases, the attachment mechanism 702 includes a gel layer disposed between the skin of the subject 703 and

In various cases, the housing 704 of the chest-mounted apparatus 700 is configured to substantially conform with the skin of the subject 703. For instance, the housing 704 may include a flexible material that is configured to minimize gaps (e.g., including air) between the housing 704 and the skin of the chest of the subject 703. In some implementations, the housing 706 includes a material that facilitates the administration of chest compressions to the subject 703. For instance, the housing 706 may include a material with a relatively high coefficient of friction (e.g., 0.5 or greater) to minimize slippage between the housing 706 and a compressor administering the chest compressions. For instance, the housing 706 includes a silicone, a polyester, a polyimide, polyether ether ketone (PEET), polyethylene terephthalate (PET), polystyrene, polyethylene, or any combination thereof.

The chest-mounted apparatus 700 includes a power source 708 configured to supply power to various components of the chest-mounted apparatus 700. In various implementations, the power source 708 is portable. For instance, the power source 708 includes at least one rechargeable (e.g., lithium-ion) battery. In some cases, the power source 708 supplies electrical energy to one or more elements of the chest-mounted apparatus 700 described herein.

In some cases, the chest-mounted apparatus 700 includes at least one input device 710. In various examples, the input device(s) 710 is configured to receive an input signal from a user 712, who may be a rescuer treating the subject 703. Examples of the input device(s) 710 include, for instance, at a keypad, a cursor control, a touch-sensitive display, a voice input device (e.g., a microphone), a haptic feedback device (e.g., a gyroscope), or any combination thereof. In various implementations, the processor(s) 714 generate the control signal based on the input signal.

According to some examples, the input device(s) 710 include one or more sensors. The sensor(s), for example, is configured to detect one or more parameters of the subject 703 and/or the chest-mounted apparatus 700. In various cases, the input device(s) 710 include one or more accelerometers. In some examples, multiple accelerometers among the input device(s) 710 are configured to detect accelerations at multiple positions along the chest of the subject 703, such as positions on either side of the midline of the subject 703. The input device(s) 710 may include electrodes, a thermometer, an oximetry sensor, a blood pressure sensor, an ultrasound transducer, a microphone, a gas sensor, a gyroscope, pressure sensors, or any combination thereof.

The chest-mounted apparatus 700 may include at least one processor 714. In various implementations, the input device(s) 710 is communicatively coupled to the processor(s) 714. The processor(s) 714 is configured to activate the input device(s) 710, such as during particular times (e.g., during respiration cycles, ventilation cycles, chest compressions, or the like). In some aspects, the processor(s) 714 analyzes data indicative of the parameter(s) detected by the input device(s) 710.

In various implementations, the chest-mounted apparatus 700 includes at least one transceiver 716 configured to communicate with at least one external device 718 over one or more communication networks 720. Any communication network described herein can be included in the communication network(s) 720 illustrated in FIG. 7. The external device(s) 718, for example, includes at least one of a monitor-defibrillator, an AED, an ECMO device, a ventilation device, a patient monitor, a mobile phone, a server, or a computing device. In some implementations, the transceiver(s) 716 is configured to communicate with the external device(s) 718 by transmitting and/or receiving signals wirelessly. For example, the transceiver(s) 716 includes a NIC, a network adapter, a LAN adapter, or a physical, virtual, or logical address to connect to the various external devices and/or systems. In various examples, the transceiver(s) 716 includes any sort of wireless transceivers capable of engaging in wireless communication (e.g., RF communication). For example, the communication network(s) 720 includes one or more wireless networks that include a 3GPP network, such as an LTE RAN (e.g., over one or more LTE bands), an NR RAN (e.g., over one or more NR bands), or a combination thereof. In some cases, the transceiver(s) 716 includes other wireless modems, such as a modem for engaging in WI-FI®, WIGIG®, WIMAX®, BLUETOOTH®, or infrared communication over the communication network(s) 720. In some aspects, the transceiver(s) 716 is configured to communicate with the external device(s) 718 via the transmission of one or more ultrasonic signals. The signals, in various cases, encode data in the form of data packets, datagrams, or the like.

In various cases, the processor(s) 714 analyzes data encoded in the signals received from the external device(s) 718. For instance, the signals include indications of encryption keys, other parameters of the subject 102, and so on.

The chest-mounted apparatus 700 further includes at least one output device 725, in various implementations. Examples of the output device(s) 725 include, for instance, least one of a display (e.g., a projector, an LED screen, etc.), a speaker, a haptic output device, a printer, or any combination thereof. In some implementations, the output device(s) 725 include a screen configured to display various parameters detected by and/or reported to the chest-mounted apparatus 700, conditions of the subject 102, conditions of treatments administered to the subject 703, conditions of the chest-mounted apparatus 700, a charge level of the power source 708, other relevant information.

The chest-mounted apparatus 700 further includes memory 726. In various implementations, the memory 726 is volatile (such as random access memory (RAM)), non-volatile (such as read only memory (ROM), flash memory, etc.) or some combination of the two. The memory 726 stores instructions that, when executed by the processor(s) 714, causes the processor(s) 714 to perform various operations. In various examples, the memory 726 stores methods, threads, processes, applications, objects, modules, any other sort of executable instruction, or a combination thereof. In some cases, the memory 726 stores files, databases, or a combination thereof. In some examples, the memory 726 includes, but is not limited to, RAM, ROM, EEPROM, flash memory, or any other memory technology. In some examples, the memory 726 includes one or more of CD-ROMs, DVDs, CAM, or other optical storage, magnetic cassettes, magnetic tape, magnetic disk storage or other magnetic storage devices, or any other medium which can be used to store the desired information. In various cases, the memory 726 stores instructions, programs, threads, objects, data, or any combination thereof, that cause the processor(s) 714 to perform various functions. In various cases, the memory 726 stores one or more parameters that are detected by the chest-mounted apparatus 700 and/or reported to the chest-mounted apparatus 700.

In implementations of the present disclosure, the memory 726 also stores instructions for executing a condition detector 728. By executing the condition detector 728, the processor(s) 714 is configured to detect a physiological condition, a treatment condition, or a condition of the chest-mounted apparatus 700 using various techniques described herein. In various cases, the condition(s) are detected based on parameter(s) detected by the input device(s) 710 and/or indications of parameter(s) received from the external device(s) 718 over the communication network(s) 720. In some implementations, the condition detector 728 causes the processor(s) 714 to output indications of the condition(s) via the output device(s) 725.

The memory 726, in various cases, includes instructions for executing an encoder 730. In various cases, upon executing the encoder 730, the processor(s) 714 is configured to generate one or more data packets indicating parameter(s) detected by the input device(s) 710 and/or conditions derived by executing the condition detector 728. In various implementations, the processor(s) 714 interleave data indicating different parameters detected by the input device(s) 710 within the data packet(s). According to some examples, the processor(s) 714 encrypt the payloads of the data packet(s) using one or more encryption keys. In some aspects, an encryption key is extracted and/or derived from a communication signal transmitted by the external device(s) 718 to the transceiver(s) 716. In various implementations, the data packet(s) are encoded within at least one communication signal transmitted by the transceiver(s) 716.

### Example Clauses

The following clauses provide various implementations of the present disclosure. However, the scope of the disclosure is not limited to any of the clauses listed below.
1. A chest-mounted apparatus, including: a first accelerometer configured to detect an acceleration at a first side of a chest of a subject at a predetermined time; a second accelerometer configured to detect an acceleration at a second side of the chest of the subject at the predetermined time, the first side of the subject being divided from the second side of the subject by a midline of the subject; a processor configured to: determine that the predetermined time is in a respiration cycle of the subject; determine a difference between the acceleration at the first side of the chest and the acceleration at the second side of the chest; determine that the difference between the acceleration at the first side of the chest and the acceleration at the second side of the chest is above a threshold; in response to determining that the difference between the acceleration at the first side of the chest and the acceleration at the second side of the chest is above the threshold, predict that the subject has a pneumothorax; and generate a data packet indicating the pneumothorax and the predetermined time; a transceiver configured to transmit, to a monitor, a communication signal encoding the data packet; a battery configured to supply energy to the first accelerometer, the second accelerometer, the processor, and the transceiver; and a housing configured to be disposed on the chest of the subject and substantially enclosing the first accelerometer, the second accelerometer, the processor, and the transceiver.
2. The chest-mounted apparatus of clause 1, the communication signal being a first communication signal, wherein the transceiver is further configured to receive, from the monitor, a second communication signal indicating a capnograph of the subject, wherein the processor is configured to determine that the predetermined time is in the respiration cycle of the subject by analyzing the capnograph.
3. The chest-mounted apparatus of clause 1 or 2, further including: an oximetry sensor configured to detect a blood oxygenation of the subject, wherein the processor is configured to: predict that the subject has the pneumothorax further by determining that the blood oxygenation is below a threshold oxygenation; and generate the data packet to further indicate the blood oxygenation.
4. An apparatus, including: a first accelerometer configured to detect an acceleration at a first side of a chest of a subject; a second accelerometer configured to detect an acceleration at a second side of the chest of the subject, the first side of the subject being divided from the second side of the subject by a midline of the subject; and a processor configured to: determine a discrepancy between the acceleration at the first side of the chest and the acceleration at the second side of the chest; and determine a physiological condition or a treatment condition of the subject by analyzing the discrepancy.
5. The apparatus of clause 4, wherein the first accelerometer is configured to detect the acceleration at the first side during a respiration cycle or a ventilation cycle, wherein the second accelerometer is configured to detect the acceleration at the second side during the respiration cycle or the ventilation cycle, and wherein the physiological condition includes a pneumothorax
6. The apparatus of clause 4 or 5, wherein the treatment condition includes a position of an endotracheal tube in the subject.
7. The apparatus of any of clauses 4 to 6, wherein the first accelerometer is configured to detect the acceleration at the first side during a ventilation cycle, wherein the second accelerometer is configured to detect the acceleration at the second side during the ventilation cycle, and wherein the treatment condition includes an insufflation volume.
8. The apparatus of any of clauses 4 to 7, wherein the first accelerometer is configured to detect the acceleration at the first side during a chest compression, wherein the second accelerometer is configured to detect the acceleration at the second side during the chest compression, and wherein the treatment condition includes a depth or a position of the chest compression.
9. The apparatus of any of clauses 4 to 8, wherein the processor is further configured to generate a data packet indicating the physiological condition or the treatment condition, and wherein the apparatus further includes: a transceiver configured to transmit, to an external device, a communication signal encoding the data packet.
10.The apparatus of clause 9, wherein the data packet further indicates the acceleration at the first side of the chest and the acceleration at the second side of the chest.
11.The apparatus of clause 9 or 10, the communication signal being a first communication signal, the transceiver being further configured to receive, from the external device, a second communication signal encoding an indication of an acceleration of the external device, wherein the processor is configured to determine the physiological condition of the subject by further comparing the acceleration of the external device to the acceleration at the first side of the chest or to the acceleration at the second side of the chest, the physiological condition including independent ambulation of the subject.
12.The apparatus of any of clauses 4 to 11, further including: a physiological sensor configured to detect a physiological parameter of the subject, wherein the processor is configured to determine the physiological condition or the treatment condition of the subject further by analyzing the physiological parameter.
13.A method, including: detecting, by a chest-mounted device, an acceleration at a first side of a chest of a subject; detecting, by the chest-mounted device, an acceleration at a second side of the chest of the subject, the first side of the subject being divided from the second side of the subject by a midline of the subject; determining a discrepancy between the acceleration at the first side of the chest and the acceleration at the second side of the chest; and determining a physiological condition or a treatment condition of the subject by analyzing the discrepancy.
14.The method of clause 13, wherein detecting the acceleration at the first side of the chest includes detecting the acceleration at the first side during a respiration cycle or a ventilation cycle, wherein detecting the acceleration at the second side of the chest includes detecting the acceleration at the second side during the respiration cycle or the ventilation cycle, and wherein the physiological condition includes a pneumothorax.
15.The method of clause 13 or 14, wherein the treatment condition includes a position of an endotracheal tube in the subject.
16.The method of any of clauses 13 to 15, wherein detecting the acceleration at the first side of the chest includes detecting the acceleration at the first side during a ventilation cycle, wherein detecting the acceleration at the second side of the chest includes detecting the acceleration at the second side during the ventilation cycle, and wherein the treatment condition includes an insufflation volume.
17.The method of any of clauses 13 to 16, wherein detecting the acceleration at the first side of the chest includes detecting the acceleration at the first side during a chest compression applied to the subject, wherein detecting the acceleration at the second side of the chest includes detecting the acceleration at the second side during the chest compression, and wherein the treatment condition includes a depth or a position of the chest compression.
18.The method of any of clauses 13 to 17, further including: generating a data packet by encrypting an indication of the physiological condition or the treatment condition; and transmitting, to an external device, a communication signal encoding the data packet.
19.The method of clause 18, the communication signal being a first communication signal, the method further including: receiving, from the external device, a second communication signal encoding an indication of an acceleration of the external device, wherein determining the physiological condition of the subject further includes comparing the acceleration of the external device to the acceleration at the first side of the chest or to the acceleration at the second side of the chest, the physiological condition including independent ambulation of the subject.
20.The apparatus of any of clauses 4 to 19, further including: detecting a physiological parameter of the subject, wherein determining the physiological condition or the treatment condition of the subject further includes analyzing the physiological parameter.
21.A chest-mounted apparatus, including: an accelerometer configured to detect an acceleration of a chest of a subject at a predetermined time; an oximetry sensor including: a first light source configured to emit light having a first wavelength into the chest of the subject; a second light source configured to emit light having a second wavelength into the chest of the subject; and a light detector configured to: detect, at the predetermined time, scatter of the light having the first wavelength from blood in the chest of the subject; and detect, at the predetermined time, scatter of the light having the second wavelength from blood in the chest of the subject; a processor configured to: determine a blood oxygenation of the subject by analyzing the scatter of the light having the first wavelength and the scatter of the light having the second wavelength; determine that the subject has spontaneous circulation by: determining that the acceleration at the predetermined time is below a first threshold; and determining that the blood oxygenation of the subject at the predetermined time is above a second threshold; and generate a data packet by encrypting an indication of the predetermined time and an indication of the spontaneous circulation; a transceiver configured to transmit, to a monitor, a communication signal encoding the data packet; a battery configured to supply energy to the accelerometer, the oximetry sensor, the processor, and the transceiver; and a housing configured to be disposed on the chest of the subject and substantially enclosing the accelerometer, the oximetry sensor, and the transceiver.
22.The chest-mounted apparatus of clause 21, the communication signal being a first communication signal, wherein the transceiver is further configured to receive, from the monitor, a second communication signal, and wherein the processor is further configured to generate an encryption key by analyzing the second communication signal, and wherein the processor is configured to generate the data packet by encrypting, using the encryption key, the indication of the predetermined time and the indication of the spontaneous circulation.
23.The chest-mounted apparatus of clause 21 or 22, wherein the processor is configured to generate the data packet further by encrypting an indication of the blood oxygenation.
24.An apparatus, including: a first sensor configured to detect a first parameter of a subject or the apparatus at a predetermined time; a second sensor configured to detect a second parameter of the subject or the apparatus at the predetermined time; a transceiver configured to transmit a communication signal encoding a data packet, the data packet including an indication of the predetermined time, an indication of the first parameter, and an indication of the second parameter; and a housing configured to be disposed on the subject and substantially enclosing the first sensor, the second sensor, and the transceiver.
25.The apparatus of clause 24, wherein the first sensor or the second sensor includes electrodes, a thermometer, an oximetry sensor, a blood pressure sensor, an ultrasound transducer, a microphone, a gas sensor, a gyroscope, or an accelerometer.
26.The apparatus of clause 24 or 25, wherein the first parameter is different than the second parameter.
27.The apparatus of any of clauses 24 to 26, wherein the first parameter or the second parameter includes an electrocardiogram (ECG), an EEG, an EMG, a temperature, a blood oxygenation, a blood pressure, a blood flow parameter, an airway parameter, a sound, or an acceleration.
28.The apparatus of any of clauses 24 to 27, wherein the communication signal includes an electromagnetic signal or an ultrasonic signal.
29.The apparatus of any of clauses 24 to 28, wherein the indication of the first parameter and the indication of the second parameter are interleaved in the data packet.
30.The apparatus of clause 24, further including: a processor configured to generate the data packet by encrypting, using an encryption key, the indication of the first parameter and the indication of the second parameter.
31.The apparatus of clause 30, the communication signal being a first communication signal, wherein the transceiver is further configured to receive, from an external device, a second communication signal, and wherein the processor is configured to generate the encryption key by analyzing the second communication signal.
32.The apparatus of any of clauses 24 to 31, wherein the first parameter includes an acceleration of a chest of the subject, wherein the second parameter includes a blood flow parameter of the subject, and wherein the apparatus further includes a processor configured to: determine that the acceleration of the chest of the subject at the predetermined time is below a first threshold; in response to determining that the acceleration of the chest of the subject at the predetermined time is below the first threshold, determine that the blood flow parameter of the subject at the predetermined time is above a second threshold; in response to determining that the blood flow parameter of the subject at the predetermined time is above the second threshold, determine that the subject has spontaneous circulation; and generate the data packet to further includes an indication that the subject has spontaneous circulation.
33.A method, including: detecting a first parameter of a subject or of a chest-mounted apparatus at a predetermined time; detecting a second parameter of the subject or the chest-mounted apparatus at the predetermined time; generating a data packet including an indication of the predetermined time, an indication of the first parameter, and the indication of the second parameter; and transmitting, to an external device, a communication signal encoding the data packet.
34.The method of clause 33, wherein the first parameter is different than the second parameter.
35.The method of clause 33 or 34, wherein the first parameter or the second parameter includes an electrocardiogram (ECG), an EEG, an EMG, a temperature, a blood oxygenation, a blood pressure, a blood flow parameter, an airway parameter, a sound, or an acceleration.
36.The method of any of clauses 33 to 35, wherein the communication signal includes an electromagnetic signal or an ultrasonic signal.
37.The method of any of clauses 33 to 36, wherein generating the data packet includes interleaving the indication of the first parameter and the indication of the second parameter.
38.The method of any of clauses 33 to 37, wherein generating the data packet includes encrypting, using an encryption key, the indication of the predetermined time, the indication of the first parameter, and the indication of the second parameter.
39.The method of clause 38, the communication signal being a first communication signal, the method further including: receiving, from an external device, a second communication signal; and generating the encryption key by analyzing the second communication signal.
40.The method of any of clauses 33 to 39, wherein the first parameter includes an acceleration of a chest of the subject, wherein the second parameter includes a blood flow parameter of the subject, and wherein the method further includes: determining that the acceleration of the chest of the subject at the predetermined time is below a first threshold; in response to determining that the acceleration of the chest of the subject at the predetermined time is below the first threshold, determining that the blood flow parameter of the subject at the predetermined time is above a second threshold; and in response to determining that the blood flow parameter of the subject at the predetermined time is above the second threshold, determining that the subject has spontaneous circulation, wherein the data packet further includes an indication that the subject has spontaneous circulation.

### Conclusion

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A chest-mounted apparatus, comprising:
a first accelerometer configured to detect an acceleration at a first side of a chest of a subject at a predetermined time;
a second accelerometer configured to detect an acceleration at a second side of the chest of the subject at the predetermined time, the first side of the subject being divided from the second side of the subject by a midline of the subject;
a processor configured to:
determine that the predetermined time is in a respiration cycle of the subject;
determine a difference between the acceleration at the first side of the chest and the acceleration at the second side of the chest;
determine that the difference between the acceleration at the first side of the chest and the acceleration at the second side of the chest is above a threshold;
in response to determining that the difference between the acceleration at the first side of the chest and the acceleration at the second side of the chest is above the threshold, predict that the subject has a pneumothorax; and
generate a data packet indicating the pneumothorax and the predetermined time;
a transceiver configured to transmit, to a monitor, a communication signal encoding the data packet;
a battery configured to supply energy to the first accelerometer, the second accelerometer, the processor, and the transceiver; and
a housing configured to be disposed on the chest of the subject and substantially enclosing the first accelerometer, the second accelerometer, the processor, and the transceiver.

2. The chest-mounted apparatus of claim 1, the communication signal being a first communication signal, wherein the transceiver is further configured to receive, from the monitor, a second communication signal indicating a capnograph of the subject,
wherein the processor is configured to determine that the predetermined time is in the respiration cycle of the subject by analyzing the capnograph.

3. The chest-mounted apparatus of claim 1, further comprising:
an oximetry sensor configured to detect a blood oxygenation of the subject, wherein the processor is configured to:
predict that the subject has the pneumothorax further by determining that the blood oxygenation is below a threshold oxygenation; and
generate the data packet to further indicate the blood oxygenation.

4. An apparatus, comprising:
a first accelerometer configured to detect an acceleration at a first side of a chest of a subject;
a second accelerometer configured to detect an acceleration at a second side of the chest of the subject, the first side of the subject being divided from the second side of the subject by a midline of the subject; and
a processor configured to:
determine a discrepancy between the acceleration at the first side of the chest and the acceleration at the second side of the chest; and
determine a physiological condition or a treatment condition of the subject by analyzing the discrepancy.

5. The apparatus of claim 4, wherein the first accelerometer is configured to detect the acceleration at the first side during a respiration cycle or a ventilation cycle,
wherein the second accelerometer is configured to detect the acceleration at the second side during the respiration cycle or the ventilation cycle, and
wherein the physiological condition comprises a pneumothorax.

6. The apparatus of claim 4, wherein the treatment condition comprises a position of an endotracheal tube in the subject.

7. The apparatus of claim 4, wherein the first accelerometer is configured to detect the acceleration at the first side during a ventilation cycle,
wherein the second accelerometer is configured to detect the acceleration at the second side during the ventilation cycle, and
wherein the treatment condition comprises an insufflation volume.

8. The apparatus of claim 4, wherein the first accelerometer is configured to detect the acceleration at the first side during a chest compression,
wherein the second accelerometer is configured to detect the acceleration at the second side during the chest compression, and
wherein the treatment condition comprises a depth or a position of the chest compression.

9. The apparatus of claim 4, wherein the processor is further configured to generate a data packet indicating the physiological condition or the treatment condition, and
wherein the apparatus further comprises:
a transceiver configured to transmit, to an external device, a communication signal encoding the data packet.

10. The apparatus of claim 9, wherein the data packet further indicates the acceleration at the first side of the chest and the acceleration at the second side of the chest.

11. The apparatus of claim 9, the communication signal being a first communication signal, the transceiver being further configured to receive, from the external device, a second communication signal encoding an indication of an acceleration of the external device,
wherein the processor is configured to determine the physiological condition of the subject by further comparing the acceleration of the external device to the acceleration at the first side of the chest or to the acceleration at the second side of the chest, the physiological condition comprising independent ambulation of the subject.

12. The apparatus of claim 4, further comprising:
a physiological sensor configured to detect a physiological parameter of the subject,
wherein the processor is configured to determine the physiological condition or the treatment condition of the subject further by analyzing the physiological parameter.

13. A method, comprising:
detecting, by a chest-mounted device, an acceleration at a first side of a chest of a subject;
detecting, by the chest-mounted device, an acceleration at a second side of the chest of the subject, the first side of the subject being divided from the second side of the subject by a midline of the subject;
determining a discrepancy between the acceleration at the first side of the chest and the acceleration at the second side of the chest; and
determining a physiological condition or a treatment condition of the subject by analyzing the discrepancy.

14. The method of claim 13, wherein detecting the acceleration at the first side of the chest comprises detecting the acceleration at the first side during a respiration cycle or a ventilation cycle,
wherein detecting the acceleration at the second side of the chest comprises detecting the acceleration at the second side during the respiration cycle or the ventilation cycle, and
wherein the physiological condition comprises a pneumothorax.

15. The method of claim 13, wherein the treatment condition comprises a position of an endotracheal tube in the subject.

16. The method of claim 13, wherein detecting the acceleration at the first side of the chest comprises detecting the acceleration at the first side during a ventilation cycle,
wherein detecting the acceleration at the second side of the chest comprises detecting the acceleration at the second side during the ventilation cycle, and
wherein the treatment condition comprises an insufflation volume.

17. The method of claim 13, wherein detecting the acceleration at the first side of the chest comprises detecting the acceleration at the first side during a chest compression applied to the subject,
wherein detecting the acceleration at the second side of the chest comprises detecting the acceleration at the second side during the chest compression, and
wherein the treatment condition comprises a depth or a position of the chest compression.

18. The method of claim 13, further comprising:
generating a data packet by encrypting an indication of the physiological condition or the treatment condition; and
transmitting, to an external device, a communication signal encoding the data packet.

19. The method of claim 18, the communication signal being a first communication signal, the method further comprising:
receiving, from the external device, a second communication signal encoding an indication of an acceleration of the external device,
wherein determining the physiological condition of the subject further comprises comparing the acceleration of the external device to the acceleration at the first side of the chest or to the acceleration at the second side of the chest, the physiological condition comprising independent ambulation of the subject.

20. The apparatus of claim 4, further comprising:
detecting a physiological parameter of the subject,
wherein determining the physiological condition or the treatment condition of the subject further comprises analyzing the physiological parameter.
